(19) United States
(12) Patent Application Publication
(10) Pub. No.: US 2026/0034055 A1
(43) Pub. Date: Feb. 5, 2026

(54) BIOCOMPATIBLE COMPOUND IMPLANTATION FOR DISTRACTION HISTOGENESIS

(71) Applicant: BioDynamik, Inc., Lake Forest, CA (US)

(72) Inventors: Emmon Johnny Chen, Lake Forest, CA (US); Shanbao Cheng, Lake Forest, CA (US)

(73) Assignee: BioDynamik, Inc., Lake Forest, CA (US)

(21) Appl. No.: 19/287,760

(22) Filed: Jul. 31, 2025

Related U.S. Application Data
(60) Provisional application No. 63/677,610, filed on Jul. 31, 2024.

(51) Int. Cl. A61K 9/00; A61K 9/06; A61K 47/10; A61K 47/32

(52) U.S. Cl. CPC A61K 9/0024; A61K 9/06; A61K 47/10; A61K 47/32

(57) ABSTRACT

Compositions and methods for distraction histogenesis with a biocompatible hydrogel or bio-textile. A biocompatible hydrogel includes a polymer and a crosslinking agent. The biocompatible hydrogel reaches equilibrium with surrounding fluids in the patient from seven days to twenty-two days after implantation in the patient.

### TECHNICAL FIELD

The disclosure relates generally to systems, methods, devices, and compositions for surgical procedures, and specifically relates to the use of biocompatible compositions in surgical procedures.

### BACKGROUND

In some cases, a patient benefits from improved blood flow and tissue regeneration to aid in recovery from vascular occlusion or damage, tissue trauma, bone defects, nerve damage, and other wounds. Tissue regeneration can be a major challenge for patient care and can be particularly difficult for patients experiencing ischemic diseases, chronic wounds, diabetic foot ulcers, bone defects, and other health challenges.

Tissue regeneration is particularly difficult to trigger when treating diabetic patients, and especially when treating diabetic foot ulcers. Currently, there are few well-known successful treatments for recalcitrant diabetic foot ulcers. Ongoing research suggests there may be some success in topical therapies and oxygen treatments, but these treatments show limited success. Additionally, it can be difficult to successfully trigger bone regeneration in response to a bone defect, fracture, or other trauma. Commonly used therapies for bone regeneration include bone graft substitutes, guided bone regeneration (GBR), distraction osteogenesis (DO), and periosteal distraction.

Traditional devices for performing distraction histogenesis and angiogenesis, and specifically for performing periosteal distraction, include numerous external components that may be bothersome and painful for the patient, can be difficult and time consuming to install, require numerous component implanted into patient bone tissue, and can be difficult to adjust as time goes on. What is needed are systems, methods, and devices for performing distraction histogenesis that improve patient outcomes, reduce surgery complexity, reduce the number of percutaneous sites, and reduce the quantity of cumbersome and painful external components, and reduce the complexity of tasks that need to be performed after surgery.

In view of the foregoing, disclosed herein are systems, methods, compositions, and devices for improved distraction histogenesis surgical techniques. The systems, methods, composition, and devices described herein include the use of biocompatible compositions to initiate the separation of tissue for purposes of tissue regeneration.

### BRIEF DESCRIPTION OF THE DRAWINGS

Non-limiting and non-exhaustive implementations of the present disclosure are described with reference to the following figures, wherein like reference numerals refer to like parts throughout the various views unless otherwise specified. Advantages of the present disclosure will become better understood with regard to the following description and accompanying drawings where:

FIGS. 1A and 1B are schematic illustrations of a method for providing a hydrogel in a distraction histogenesis surgical procedure;

FIGS. 2A and 2B are schematic illustrations of a method for providing a hydrogel in a distraction histogenesis surgical procedure;

FIGS. 3A and 3B are schematic illustrations of a method for providing a bio-textile in a distraction histogenesis surgical procedure;

FIG. 4 is a schematic illustration of a process flow for expanding a hydrogel after insertion during a distraction histogenesis surgical procedure;

FIG. 5 is a schematic illustration of components of a hydrogel to be utilized in a distraction histogenesis surgical procedure;

FIG. 6A is an image of a system for inserting a hydrogel underneath a periosteum for a distraction histogenesis procedure;

FIG. 6B is an image of a system for inserting a hydrogel underneath a periosteum for a distraction histogenesis procedure, and specifically illustrates timed expansion of the hydrogel;

FIGS. 6C and 6D are schematic illustrations of a hydrogel comprising a plate geometry;

FIGS. 6E and 6F are schematic straight-on side illustrations of a hydrogel comprising a polygonal geometry;

FIGS. 6G and 6H are schematic illustrations of a hydrogel system comprising an expansion bag;

FIG. 7 is a schematic flow chart diagram of a method for performing a periosteum distraction procedure with a hydrogel;

FIG. 8 is a schematic flow chart diagram of a method for performing a distraction histogenesis procedure; and

FIG. 9 is a schematic illustration of a process for performing a periosteal distraction procedure with a hydrogel and/or bio-textile.

### DETAILED DESCRIPTION

Described herein are systems, methods, compositions, and devices for triggering tissue regeneration. The disclosures herein may be implemented to trigger regeneration of bone tissue, vascular tissue, skin tissue, and other tissue types. The techniques described herein may specifically be utilized to treat chronic wounds, diabetic foot ulcers, traumatic wounds, and bone defects by triggering the growth of new tissue.

Distraction histogenesis is a biological principle and surgical technique that involves the gradual, controlled separation of living tissues to stimulate the formation of new tissue in the gap created between the separated segments. The technique is based on the tension-stress law, which states that slow, steady mechanical tension applied to living tissues stimulates cellular regeneration and new tissue formation. Rather than simply stretching existing tissue, the controlled distraction triggers the body's natural healing and regenerative processes to create entirely new tissue. Distraction histogenesis may be utilized for bone lengthening, deformity correction, bone defect reconstruction, soft tissue applications, craniofacial surgery, and other implementations.

Periosteal distraction is a type of distraction histogenesis. Periosteal distraction is a surgical technique that involves the gradual, controlled separation of the periosteum from the underlying bone surface to stimulate blood vessel formation. Periosteal distraction works by creating a controlled injury to the periosteum and then gradually pulling the periosteum away from the bone cortex using mechanical devices or other spaces. This creates a space between the periosteum and bone that fills with new tissue through several biological processes. Periosteal distraction may cause periosteal activation, wherein osteoprogenitor cells are stimulated to proliferate and differentiate into osteoblasts, and may further cause angiogenesis to develop new blood vessels to supply developing tissue. In some cases, periosteal distraction may also result in new bone growth through intramembranous ossification and/or endochondral ossification.

Conventional techniques for periosteal distraction and/or distraction histogenesis have several drawbacks and are known to cause excessive patient trauma. Conventional devices typically have complex hardware, limited precision, and are difficult to use in cases needed multidirectional correction. Additionally, conventional devices cause significant pain and discomfort for the patient, introduce psychological trauma due to bulky, visible hardware, may cause sleep disturbances for the patient, and may cause significant mobility restrictions for the patient. Additionally, such conventional devices are known to cause infection and wound complications, through pin site infections, wound healing issues, and chronic drainage.

Conventional periosteal distraction techniques include making a surgical incision to expose the bone and periosteum. A device, such as a distractor, is then placed to gradually lift the periosteum away from the bone. Over time, the distractor is adjusted to slowly separate the periosteum from the underlying bone. This slow and controlled process stimulates the periosteum to generate new bone tissue in the gap. As the periosteum is lifted, new bone forms in the space created. This process may take several week to months, depending on the desired amount of new bone growth. Once the desired amount of bone has formed, the distraction device is left in place to allow the new bone to harden and consolidate.

What is needed are improved systems, methods, compositions, and devices for distraction histogenesis that reduce the risks of infections and device complications. In view of the foregoing, described herein systems, methods, and devices for initiating tissue growth through distraction histogenesis. Specifically described herein are compositions and methods for providing a hydrogel that is optimized to expand slowly and steadily to provide a space between tissues, and then trigger the body to generate new tissues. In some cases, the hydrogels described herein may be utilized in combination with a mechanical device configured to provide slow and steady separation between tissue types.

The systems, methods, devices, and compositions described herein may be used to trigger the growth of numerous tissue types, including soft tissues and bone tissues. Specifically, the techniques described herein may be utilized to treat chronic wounds, diabetic foot ulcers, soft tissue trauma, and bone defects.

The distraction histogenesis techniques described herein may be utilized to treat bone defects on any bone in the body by raising systemic growth factor levels. One or more of the devices described herein can be coupled to many different bones in the body, because the devices are internal and implantable.

The distraction histogenesis techniques described herein may further be utilized to treat soft tissue injuries, including chronic wounds, traumatic wounds, diabetic foot ulcers, and other injuries. The devices described herein allow for targeted treatment of soft tissue injuries in virtually any area of the body. For example, a patient presenting with sacral pressure ulcers could receive this technique and device in the pelvis which could localize the accelerated biologic activity.

In the following description of the disclosure, reference is made to the accompanying drawings, which form a part hereof, and in which is shown by way of illustration specific implementations in which the disclosure may be practiced. It is understood that other implementations may be utilized, and structural changes may be made without departing from the scope of the disclosure.

Before the structures, systems, methods, and compositions described herein are disclosed, it is to be understood that this disclosure is not limited to the particular structures, configurations, process steps, and materials disclosed herein as such structures, configurations, process steps, and materials may vary somewhat. It is also to be understood that the terminology employed herein is used for the purpose of describing particular embodiments only and is not intended to be limiting since the scope of the disclosure will be limited only by the appended claims and equivalents thereof.

In describing and claiming the subject matter of the disclosure, the following terminology will be used in accordance with the definitions set out below.

As used herein, the terms “comprising,” “including,” “containing,” “characterized by,” and grammatical equivalents thereof are inclusive or open-ended terms that do not exclude additional, unrecited elements or method steps.

As used herein, the phrase “consisting of” and grammatical equivalents thereof exclude any element, step, or ingredient not specified in the claim.

As used herein, the phrase “consisting essentially of” and grammatical equivalents thereof limit the scope of a claim to the specified ingredients, materials, or steps and those that do not materially affect the basic and novel characteristic or characteristics of the claimed disclosure.

As used herein, “effective amount” means an amount of an ingredient or a component of the product that is nontoxic, but sufficient to provide the desired effect and performance at a reasonable benefit/risk ratio attending any product. For example, an effective amount of a pharmaceutical, vitamin, supplement, or other compound/composition is an amount sufficient to provide a desired result, prevent a deficiency, and/or to reduce the incidence of some adverse effects.

Unless defined otherwise, all technical and scientific terms used herein have the same meanings as commonly understood by one of ordinary skill in the art to which this disclosure pertains and belongs.

Reference will now be made in detail to the exemplary embodiments, examples of which are illustrated in the accompanying drawings. Wherever possible, the same reference numbers are used throughout the drawings to refer to the same or like parts. It is further noted that elements disclosed with respect to particular embodiments are not restricted to only those embodiments in which they are described. For example, an element described in reference to one embodiment or figure, may be alternatively included in another embodiment or figure regardless of whether or not those elements are shown or described in another embodiment or figure. In other words, elements in the figures may be interchangeable between various embodiments disclosed herein, whether shown or not.

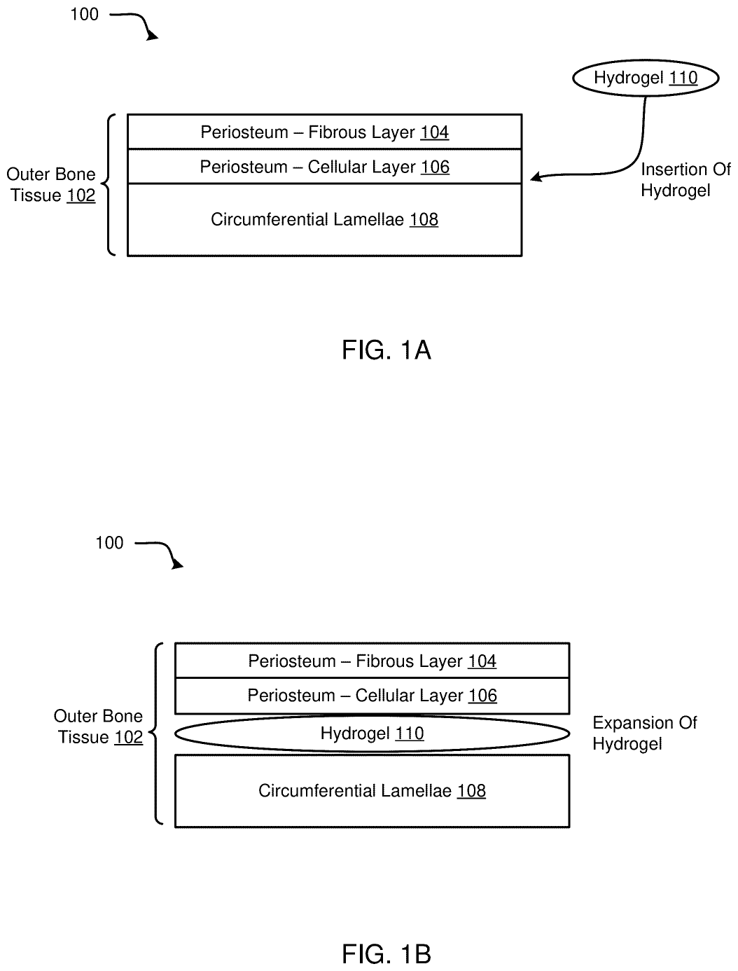

Referring now to the figures, FIGS. 1A and 1B are schematic illustrations of a method 100 for periosteal distraction. The method 100 is a specialized form of distraction histogenesis that is implemented to trigger tissue growth. The method 100 is implemented on the outer bone tissue 102 of a patient, which includes at least the periosteum 104, 106 and circumferential lamellae 108 of the bone. The periosteum includes a fibrous periosteum layer 104 and a cellular periosteum layer 106. The method 100 includes insertion of a hydrogel 110 in between the cellular periosteum layer 106 and the circumferential lamellae 108. As shown in FIG. 1B, after the hydrogel 110 is inserted, the hydrogel may expand to create an artificial separation between the periosteum 104, 106 and the circumferential lamellae 108.

The method 100 may specifically include providing anesthesia to the patient. The anesthesia may include local anesthesia, epidural anesthesia, general anesthesia, and so forth. The method 100 may include incising the patient's skin at approximately the location where the hydrogel 110 will be placed. The method 100 may include inserting a periosteal elevator or periosteal separator to create a subperiosteal tunnel. The method 100 may include opening the periosteum of the patient around the incision point. The method 100 may then include inserting the hydrogel 110 under the patient's periosteum such that the hydrogel 110 resides in between the periosteum layer 104, 106 and circumferential lamellae 108 layer of the patient's bone.

The hydrogel 110 is a porous permeable solid that is water insoluble and comprises a three-dimensional network of polymers capable of absorbing and retaining fluids while maintaining structural integrity. The hydrogel 110 is non-toxic and well-tolerated by living tissue to ensure adequate biocompatibility with surrounding tissue. The hydrogel 110 may include a natural polymer, a synthetic polymer, or a hybrid system comprising each of a natural polymer and a synthetic polymer.

In some cases, it is desirable that the hydrogel 110 exclusively or predominantly includes natural polymers that may provide greater biocompatibility, biodegradability, antibiotic effects, antifungal effects, and improve regeneration of nearby tissue.

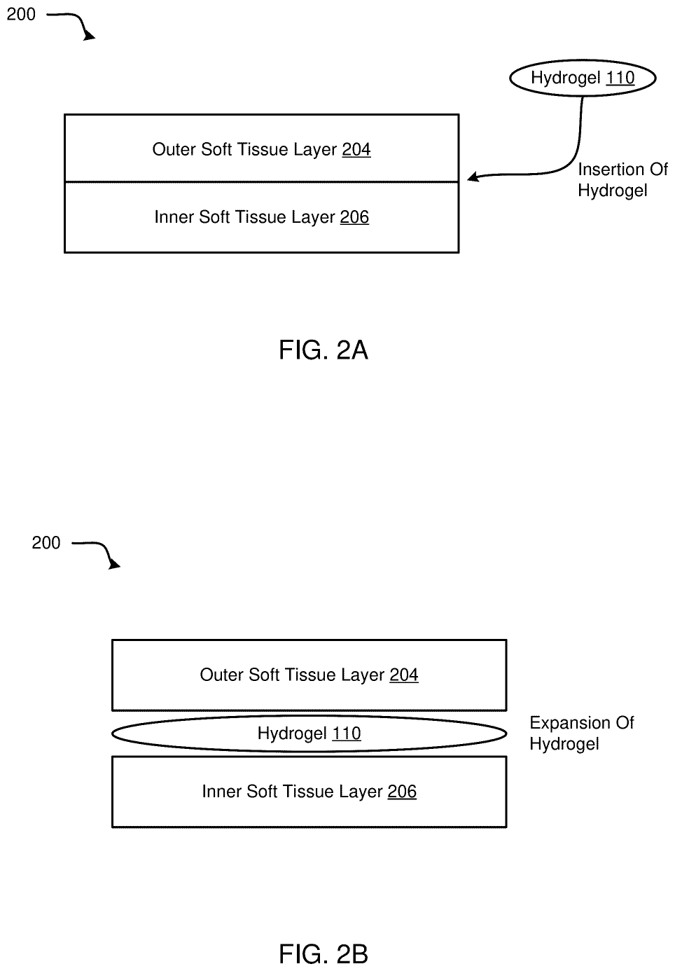

FIGS. 2A and 2B are schematic illustrations of a method 200 for distraction histogenesis that is implemented to trigger growth of soft tissues, such as blood vessels, lymph vessels, muscles, tendons, ligaments, fat, fibrous tissue, nerves, cartilage, fascia, and synovial membranes.

Like the method 100 for periosteal distraction described in connection with FIGS. 1A and 1B, the method 200 for distraction histogenesis illustrated in FIGS. 2A and 2B operates by inserting a hydrogel 110 in between tissues. The method 200 includes inserting a hydrogel 110 in between an outer soft tissue layer 204 and an inner soft tissue layer 206. The hydrogel 110 then expands after insertion, and thereby separates the outer soft tissue layer 204 from the inner soft tissue layer 206. This triggers a healing response in the body to generate new tissue.

The outer soft tissue layer 204 and the inner soft tissue layer 206 may comprise any applicable soft tissue such as, for example, blood vessels, lymph vessels, muscles, tendons, ligaments, fat, fibrous tissue, nerves, cartilage, fascia, and synovial membranes.

In some cases, the outer soft tissue layer 204 and the inner soft tissue layer 206 comprise the same tissue type. This may specifically occur when the hydrogel 110 is inserted within a wound area that has suffered a traumatic injury or continues to suffer an ongoing chronic injury, such as a diabetic foot ulcer. In other cases, the outer soft tissue layer 204 and the inner soft tissue layer 206 comprise different tissue types.

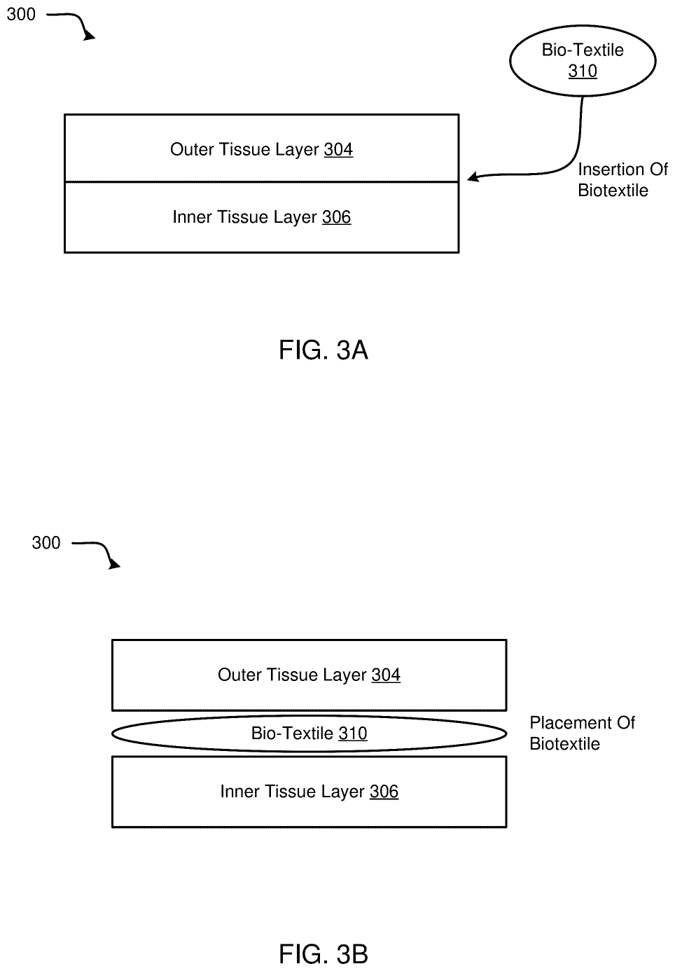

FIGS. 3A and 3B are schematic illustrations of a method 300 for distraction histogenesis that is implemented to trigger growth of tissues, such as bone tissue, blood vessels, lymph vessels, muscles, tendons, ligaments, fat, fibrous tissue, nerves, cartilage, fascia, and synovial membranes. The method 300 may be utilized for periosteal distraction similar to the method 100 described in connection with FIGS. 1A and 1B. Additionally or alternatively, the method 300 may be utilized for distraction histogenesis of soft tissues similar to the method 200 described in connection with FIGS. 2A and 2B.

The method 300 includes insertion of a bio-textile 310 in between an inner tissue layer 306 and an outer tissue layer 304. One or more of the inner tissue layer 306 or the outer tissue layer 304 may comprise a soft tissue such as, for example, blood vessels, lymph vessels, muscles, tendons, ligaments, fat, fibrous tissue, nerves, cartilage, fascia, and synovial membranes. In other use-cases, the inner tissue layer 306 may include a circumferential lamellae of a bone, and the outer tissue layer 304 may include a periosteum surrounding the bone.

The bio-textile 310 is a specialized material engineered from natural or synthetic fibers. The bio-textile 310 is designed to interact with biological systems and offers biocompatibility, porosity, and mechanical strength. The bio-textile 310 may include one or more of a mycelium-based textile, algae-derived fiber, cellulose-based textile, recombinant protein-based textile, or other type of bio-textile.

The placement of the bio-textile 310 causes separation between the inner tissue layer 306 and the outer tissue layer 304, and this in turn triggers a regenerative tissue response by the body. The method 300 may be utilized to treat soft tissue injuries, including chronic injuries, diabetic foot ulcers, traumatic injuries, and other soft tissue defects. The method 300 may further be utilized to treat bone defects.

The bio-textile 310 may include a resorbable mesh such as a polylactic acid (PLA) mesh, a polyglycolic acid (PGA) fabric, a polydioxanone (PDO) material, or a poly lactide-co-glycolide (PLGA) material. The bio-textile 310 may include a woven mesh, knitted fabric, nonwoven membrane, or braided construction. The bio-textile 310 has controlled porosity, appropriate tensile strength, flexibility, and high biocompatibility.

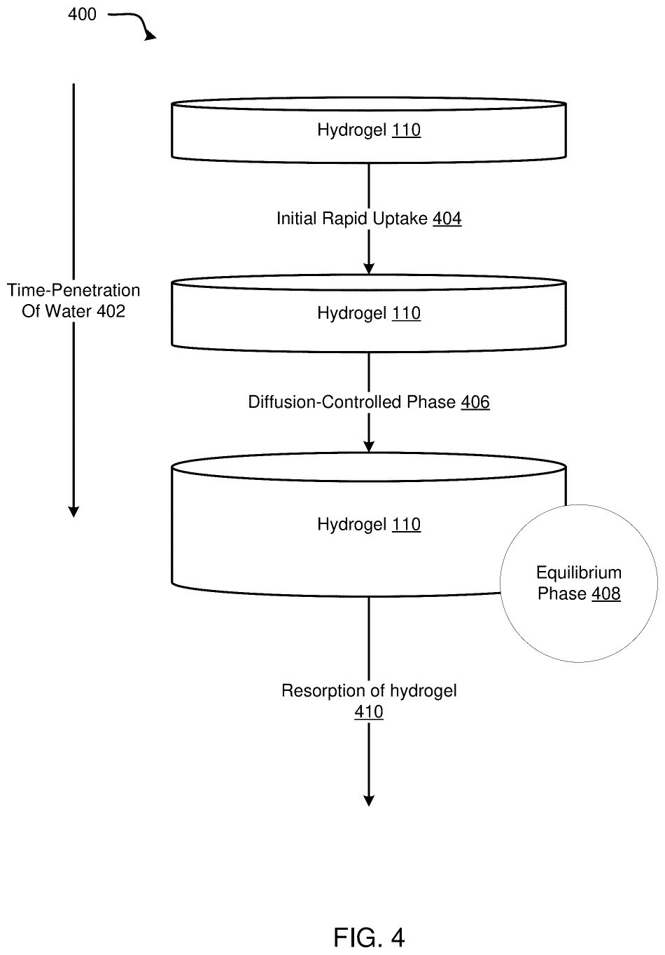

FIG. 4 is a schematic illustration of a process 400 for time-optimized expansion and resorption of the hydrogel 110. The process 400 is optimized for a successful distraction histogenesis procedure.

The process 400 includes inserting the hydrogel 110 underneath a periosteum layer of a patient. The hydrogel 110 undergoes time-penetration of water 402, and this results in swelling of the hydrogel 110. The time-penetration of water 402 includes an initial rapid uptake 404, a diffusion-controlled phase 406, and an equilibrium phase 408. The hydrogel 110 may absorb fluids already present in the patient's body and/or the hydrogel 110 may absorb additional water or other fluids placed at the surgical site by a surgeon. The hydrogel 110 eventually swells to its maximum size when it reaches the equilibrium phase 408, and ideally, this is the correct size required for successful patient outcomes in a distraction histogenesis surgical procedure. The hydrogel 110 undergoes resorption 410 wherein the patient's body breaks down and absorbs the materials of the hydrogel 110.

The time-penetration of water 402 follows several phases, including the initial rapid uptake 404, which may take minutes up to an hour to occur; the diffusion-controlled phase 406, which may take hours or days to occur; and the equilibrium phase 408, which may take days or weeks to occur. The initial rapid uptake 404 includes initial swelling that creates more pathways for further penetration, and the rate of the initial rapid uptake is governed by the surface area and initial porosity of the hydrogel 110. The diffusion-controlled phase 406 includes water diffusing through the polymer network of the hydrogel 110. The polymer network relaxes as chains become more mobile, and the rate of water uptake during this phase decreases as the hydrogel 110 approaches the equilibrium phase. In the equilibrium phase 408, the hydrogel 110 has reached maximum selling capacity and formed a stable hydrated network structure.

The rate of time-penetration of water 402 is impacted by the material properties of the hydrogel 110, including the crosslink density, polymer hydrophilicity, initial porosity, and molecular weight between crosslinks. If the hydrogel 110 has a higher density of crosslinks, then the time-penetration of water 402 will occur at a slower rate. If the hydrogel 110 has more hydrophilic molecules, then the time-penetration of water 402 will occur at a quicker rate. If the hydrogel 110 has higher porosity, then the initial rapid uptake 404 will occur at a faster rate.

The rate of time-penetration of water 402 is further impacted by the parameters of the surgical site, including the temperature, pH, ionic strength, and hydrogel geometry. The temperature is controlled by the baseline body temperature of the patient, along with additional head output due to inflammation. Higher temperatures at the surgical site will lead to quicker rates for the time-penetration of water 402 into the hydrogel 110. The geometry of the hydrogel 110 will further impact the time-penetration of water 402, such that thinner or smaller hydrogels 110 will hydrate more quickly than bulkier hydrogels 110.

The resorption 410 may occur through hydrolytic degradation, wherein water within the patient's body breaks down polymer chains in the hydrogel 110. The resorption 410 may additionally or alternatively occur through one or more of enzymatic degradation, oxidative degradation, or phagocytic resorption. The resorption 410 rate is impacted by the molecular weight of the hydrogel 110, such that a hydrogel with a higher molecular weight will degrade slower than a hydrogel 110 with a lighter molecular weight. The resorption 410 rate is impacted by the crystalline components of the hydrogel 110, such that hydrogels 110 comprising crystalline components will degrade more slowly than hydrogels 110 without crystalline components. The resorption 410 rate is impacted by the porosity of hydrogel 110, such that more porous hydrogels will degrade and be resorbed more quickly than non-porous hydrogels. The resorption 410 rate is impacted by the tissue vascularity at the surgical site, such that tissues with increased blood supply will resorb the hydrogel 110 more quickly. The resorption 410 rate is impacted by mechanical stress on the hydrogel 110, such that more force on the hydrogel 110 can lead to faster degradation and faster resorption.

The hydrogel 110 is optimized such that the resorption 410 occurs within seven to 30 days after the hydrogel 110 is implanted into the patient. If the hydrogel 110 is resorbed 406 too quickly then the patient's periosteum may not be distracted long enough for sufficient bone tissue growth, and this is a sub-optimal patient outcome. Conversely, if the hydrogel 110 is resorbed 406 too slowly then the patient may experience excessive bone tissue growth, and this is also a sub-optimal patient outcome. The hydrogel 110 may specifically be optimized to remain within the patient's body for 14 days, or for about 10 days to about 18 days.

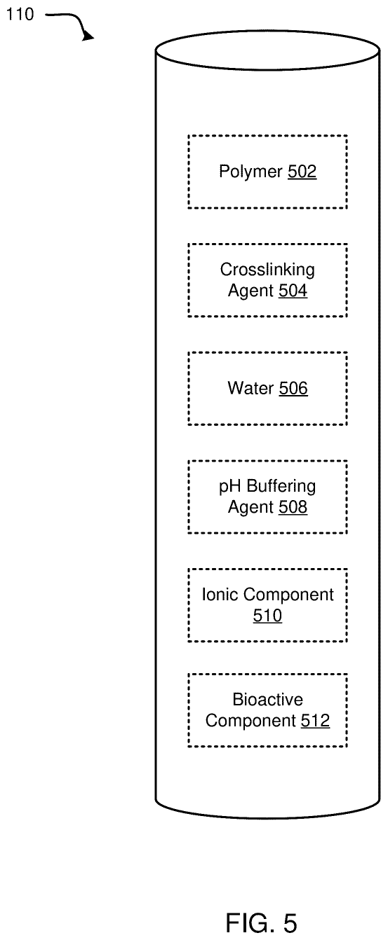

FIG. 5 is a schematic diagram illustrating components of a hydrogel 110 formed in a cylindrical geometry. The hydrogel 110 includes a polymer 502, crosslinking agent 504, and water 506. The hydrogel 110 may optionally include one or more of a pH buffering agent 508, ionic component 510, or bioactive component 512.

The hydrogel 110 includes a polymer 502. The polymer 502 provides the main structural framework for the hydrogel 110 and may include a natural or synthetic polymer. The polymer 502 determines the mechanical and chemical properties of the hydrogel 110, and may include hydrophilic molecular groups for water interaction.

The hydrogel 110 includes a crosslinking agent 504. The crosslinking agent 504 creates covalent or non-covalent bonds between polymer 502 chains. The crosslinking agent 504 controls the network density and gel strength of the hydrogel 110, and further impacts the swelling capacity and mechanical properties of the hydrogel 110.

The hydrogel 110 includes water 506, and may specifically include from about 70 vol. % to about 99 vol. % the water 506. The water 506 functions as the continuous phase and enables nutrient transport and waste transport through the hydrogel 110. The water 506 further provides the soft tissue-like properties of the hydrogel 110.

The hydrogel 110 may include a natural polymer 502. The hydrogel 110 may specifically include a polysaccharide such as one or more of alginate, chitosan, hyaluronic acid, cellulose, agarose, pectin, carrageenan, xanthan gum, guar gum, starch, or a modified starch. The hydrogel 110 may specifically include a protein such as one or more of collagen, gelatin, fibrin, silk fibroin, elastin, or albumin. The hydrogel 110 may specifically include one or more of heparin, chondroitin sulfate, or keratin.

The hydrogel 110 may include a synthetic polymer 502. The hydrogel 110 may specifically include a polyethylene glycol derivative such as one or more of polyethylene glycol (PEG), polyethylene glycol diacrylate (PEGDA), polyethylene glycol dimethacrylate (PEGDMA), or multi-arm polyethylene glycol. The hydrogel 110 may specifically include an acrylic or methacrylic polymer 502 such as one or more of polyacrylic acid (PAA), polymethacrylic acid (PMAA), poly 2-hydroxyethyl metacrylate (pHEMA), poly N-isopropylacrylamide (pNIPAM), or polyacrylamide (PAM). The hydrogel 110 may specifically include a vinyl polymer 502 such as one or more of polyvinyl alcohol (PVA), polyvinyl pyrroidone (PVP), or poly N-vinyl caprolactam. The hydrogel 110 may specifically include one or more of polyethylene oxide (PEO), pluronic, poly 2-oxazoline, poly dimethylsiloxane, polyurethanes, polycaprolactone, poly N,N-diethylacrylamide, poly dimethylaminoethyl methacrylate, or poly 4-vinylpuridine.

The hydrogel 110 includes a crosslinking agent 504. The crosslinking agent 504 may include a chemical crosslinking agent such as an aldehyde, carbodiimide, divinyl compound, or epoxy compound. The crosslinking agent 504 may include a photo-crosslinking agent such as amethacrylate group, or acrylate system. The crosslinking agent 504 may include an ionic crosslinking agent such as a multivalent cation. The crosslinking agent 504 may include an enzyme-substrate system.

The crosslinking agent 504 may include an aldehyde such as one or more of glutaraldehyde, formaldehyde, genipin, or oxidized dextran. The crosslinking agent 504 may include a carbodiimide such as one or more of 1-ethyl-3-(3-dimethylaminopropyl) carbodiimide) (EDC), dicyclohexylcarbodiimide (DCC), or N-hydroxysuccinimide (NHS). The crosslinking agent 504 may include a divinyl compound such as one or more of N,N′-methylenebisacrylamide (MBA), ethylene glycol dimethacrylate (EGDMA), or divinyl sulfone. The crosslinking agent 504 may include a methacrylate group such as one or more of glycidyl methacrylate (GMA), 2-hydroxyethyl methacrylate (HEMA), or methacrylic anhydride. The crosslinking agent 504 may include an acrylate group such as one or more of PEG diacrylate, acryloyl chloride, or N-acryloxysuccinimide. The crosslinking agent 504 may include a multivalent cation such as one or more of calcium chloride, zinc sulfate, iron chloride, or aluminum chloride.

The hydrogel 110 may optionally include a pH buffering agent 508. The pH buffering agent 508 maintains a stable pH environment for the hydrogel 110 and may be important for biocompatibility and stability in some instances. The pH buffering agent 508 may impact the swelling behavior of the hydrogel 110.

The hydrogel 110 may optionally include an ionic component 510. The ionic component 510 provides electrical conductivity and impacts osmotic pressure and swelling. The ionic component 510 may include one or more of sodium chloride, calcium chloride, or potassium phosphate.

The hydrogel 110 may optionally include a bioactive component 512. The bioactive component 512 includes one or more of a growth factor, protein, cell adhesion promoter, or antimicrobial agent. The bioactive component 512 may include a growth factor to enhance biological activity and increase tissue regeneration through bioactive signaling. The growth factor may include one or more of a bone and cartilage regeneration signaler such as BMP-2 (bone morphogenetic protein-2), BMP-7/OP-1, TGF-β (transforming growth factor-beta), or IGF-1 (insulin-like growth factor-1). The growth factor may include a vascular development growth factor such as VEGF (vascular endothelial growth factor), bFGF/fgf-2 (basic fibroblast growth factor), PDGF (platelet-derived growth factor), or angiopoietin-1. The growth factor may include a wound healing and tissue repair growth factor such as EGF (epidermal growth factor), KGF (keratinocyte growth factor), NGF (nerve growth factor), or HGF (hepatocyte growth factor).

The bioactive component 512 may include a cell adhesion promoter to improve cell attachment and spreading. The cell adhesion promoter may include an RGD peptide (arginine-glycine-aspartic acid). The RGD peptide may include, for example, a linear RGD sequence, cyclic RGD peptide, or integrin target. The cell adhesion promoter may include a fibronectin-derived sequence such as PHSRN, REDV peptide, or CS-1 peptide. The cell adhesion promoter may include a laminin-derived sequence such as a YIGSR sequence, IKVAV peptide, or PDSGR sequence. The cell adhesion promoter may include a collagen-derived sequence. The cell adhesion promoter may include a vitronectin-derived molecule, elastin-derived molecule, or hyaluronic acid binding molecule.

The bioactive component 512 may include an antimicrobial agent to prevent bacterial contamination and provide improved wound care. The antimicrobial agent may include a silver-based compound such as silver nanoparticles, silver nitrate, silver sulfadiazine, or silver-zeolite composites. The antimicrobial agent may include a copper compound such as copper nanoparticles, copper oxide, or copper sulfates. The antimicrobial agent may include a zinc-based agent such as zinc oxide nanoparticles or zinc sulfate.

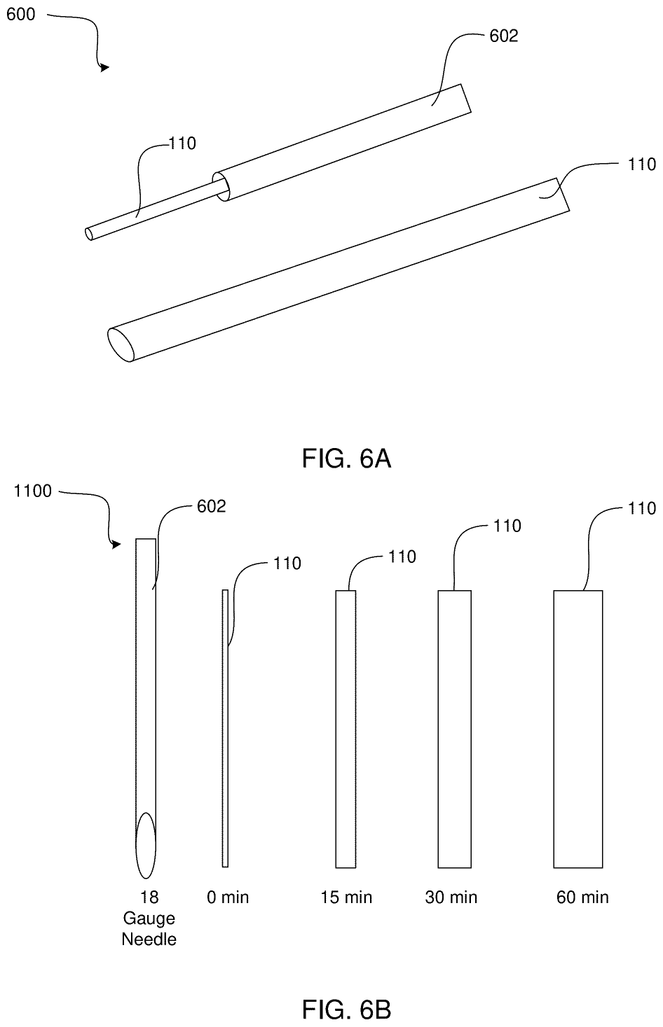

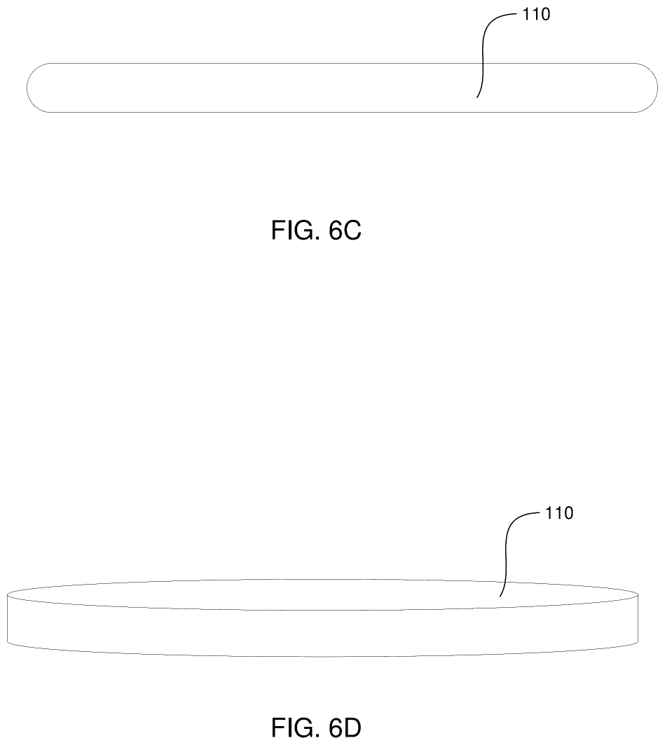

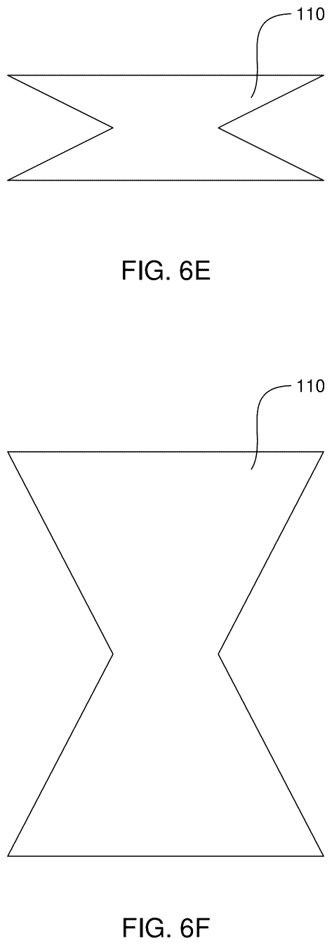

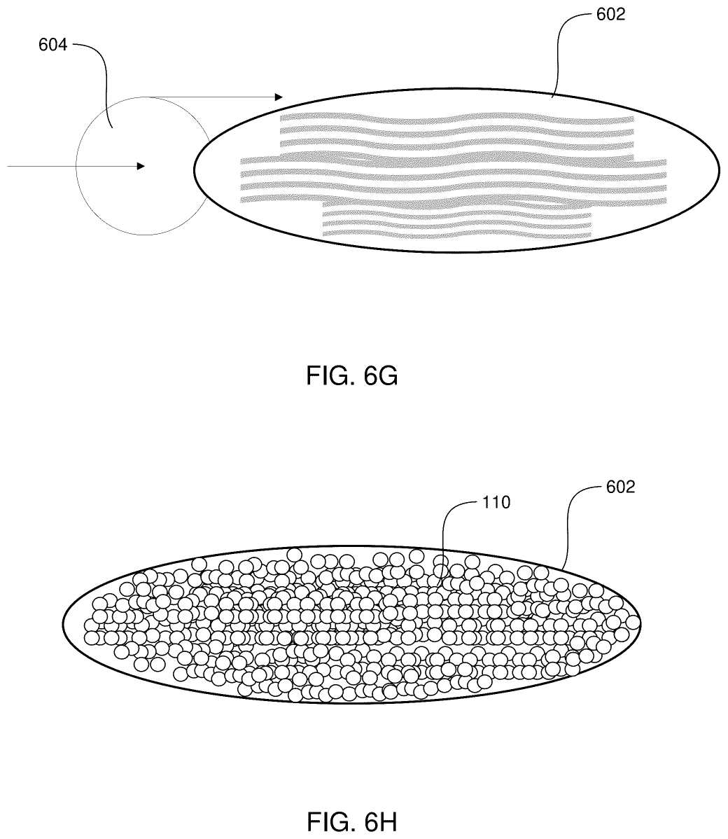

FIGS. 6A-6H are schematic illustrations of hydrogel systems that may be provided to a patient in connection with a distraction histogenesis surgical procedure. Any of the systems, compositions, or devices illustrated in FIGS. 6A-6H may be utilized in connection with any of the methods described in connection with any of FIGS. 1A-1B, 2A-2B, or 3A-3B.

FIG. 6A illustrates a hydrogel 110 disposed within a needle 602. The hydrogel 110 comprises a cylindrical geometry configured to be disposed within an interior space defined by the needle 602. The hydrogel 110 may be provided to the patient with the needle 602.

FIG. 6B illustrates the needle 602 for providing the hydrogel 110 to a patient. FIG. 6B further illustrates expansion of the hydrogel 110 over time, including its initial size at zero minutes and its gradual expansion at fifteen, thirty, and sixty minutes.

FIGS. 6C and 6D are schematic illustrations of a hydrogel 110 comprising a plate geometry. FIG. 6C is an aerial top-down view of the hydrogel 110 comprising the plate geometry with rounded edges. FIG. 6D is a perspective side view of the hydrogel 110 comprising the plate geometry with rounded edges. The hydrogel 110 illustrated in FIGS. 6C and 6D may be disposed in between two tissue types within a patient, and the geometry of the hydrogel 110 may be optimized based upon the patient's individual needs.

FIGS. 6E and 6F are schematic straight-on side illustrations of a hydrogel 110 comprising a polygonal geometry. FIG. 6E is a straight-on side view of the hydrogel 110 in a collapsed state at its minimum size. FIG. 6F is a straight-on side view of the hydrogel 110 in an expanded state at a maximum or intermediary size.

FIGS. 6G and 6H are schematic illustrations of a hydrogel system comprising an expansion bag 604. FIG. 6G is a schematic illustration of a straight on side view of the system, wherein a pump 604 is in fluid communication with the expansion bag 602. The system comprising a plurality of hydrogel 110 beads disposed within the expansion bag 602. The pump 604 may be utilized to provide measured quantities of fluid into the interior space of the expansion bag 602 to cause controlled expansion of the hydrogel 110 beads.

The system illustrated in FIGS. 6G and 6H may be utilized during a periosteal distraction surgical procedure, wherein the expansion bag 602 is sealed and placed in between two tissues of a patient. The surgeon may establish fluid communication between the expansion bag 602 and the pump 604 such that the surgeon may pump saline or another fluid into the expansion bag 602 to cause swelling of the hydrogel 110 beads. In an alternative implementation, the expansion bag 602 may be initially empty, and then filled with fluid and/or hydrogel 110 beads for controlled expansion.

In some distraction histogenesis implementations, the hydrogel 110 is configured to be disposed underneath the periosteum of the patient. In some cases, additional fluids will also be disposed underneath the periosteum of the patient to ensure there are sufficient nearby fluids for the hydrogel 110 to expand at the intended or expected rate. The hydrogel 110 expands when disposed underneath the periosteum of the patient and thereby creates an artificial space between the bone surface and the periosteum. This may cause the patient to generate new bone by gradually expanding the periosteum.

In some cases, the hydrogel 110 is not used in connection with any other devices. The hydrogel 110 may be composed of bioresorbable materials, and the hydrogel 110 alone may be utilized for the distraction histogenesis procedure. The maximum expansion of the hydrogel 110 may be optimized to ensure the hydrogel 110 creates sufficient space between the bone and the periosteum of the patient. Additionally, the hydrogel 110 may be optimized to remain within the patient for a certain duration of time to ensure the patient's body has sufficient time to initiate bone generation.

The hydrogel 110 may additionally include one or more pharmaceutical, vitamins, supplements, or other compounds disposed therein. The hydrogel 110 may thereby serve as a drug delivery matrix to elute growth factors and hasten the process of bone generation. The hydrogel 110 may include various suitable bioactive compounds to improve patient outcomes.

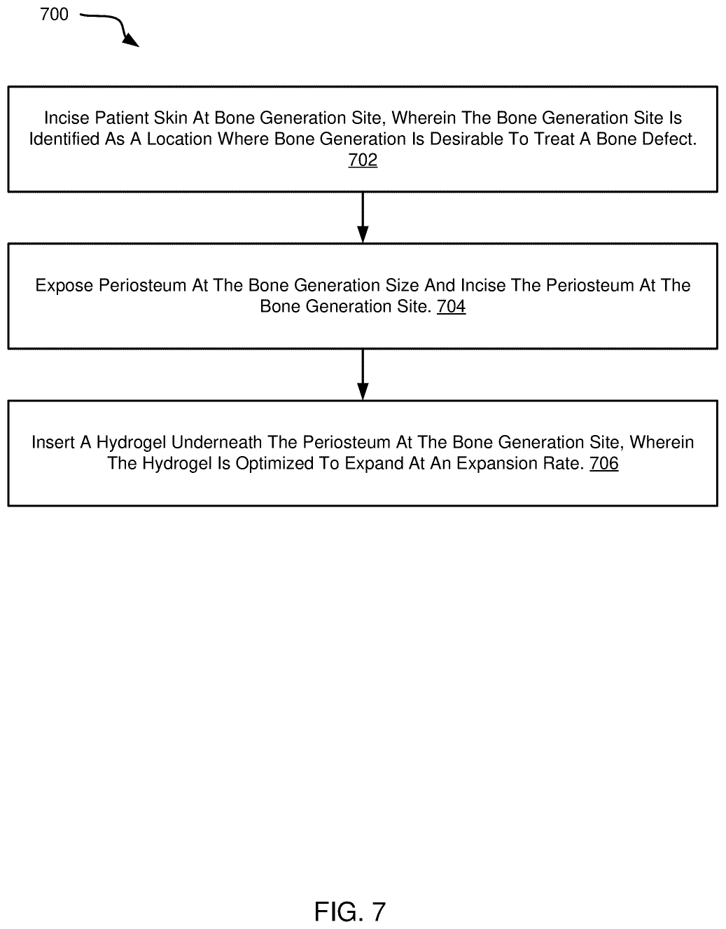

FIG. 7 is a schematic flow chart diagram of a method 700 for periosteal distraction. The method 700 includes incising at 702 patient skin at a bone generation site, wherein the bone generation site is identified as a location where bone generation is desirable to treat a bone defect in the patient. The method 700 includes exposing at 704 a periosteum of the patient at the bone generation site. The method 700 further includes incising at 704 the periosteum of the patient at the bone generation site. The method 700 includes inserting at 706 a hydrogel (see 110) underneath the periosteum at the bone generation site, wherein the hydrogel is optimized to expand at an expansion rate. The expansion rate may be optimized to prompt bone generation at the bone generation site by creating an artificial space between the bone of the patient and the periosteum of the patient.

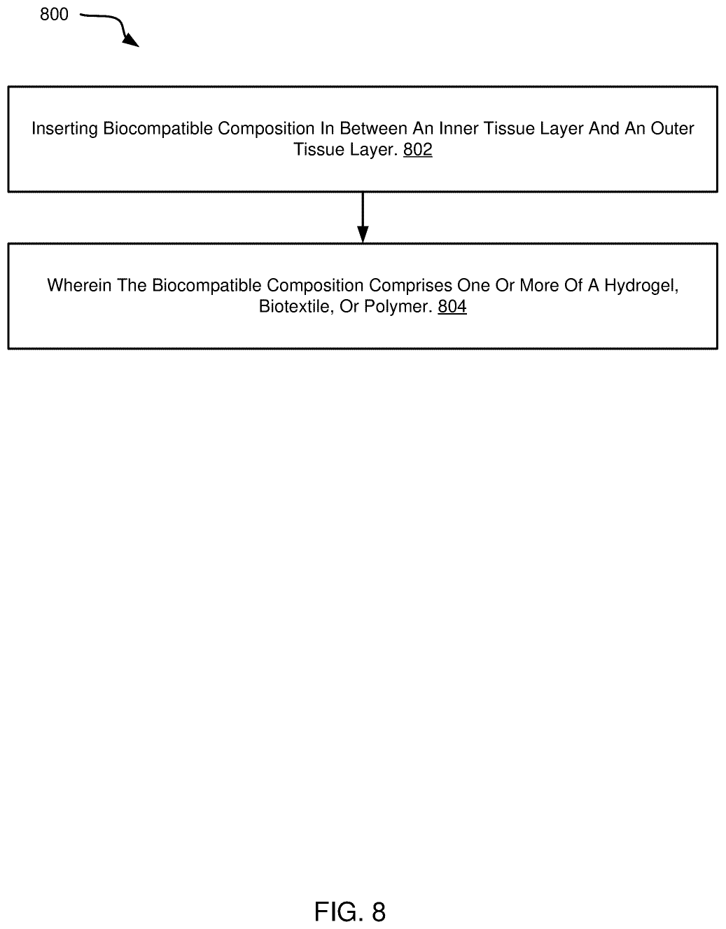

FIG. 8 is a schematic flow chart diagram of a method 800 for distraction histogenesis. The method 800 may be implemented to trigger regeneration of bone tissue or soft tissues. The method 800 includes inserting at 802 a biocompatible composition in between an inner tissue layer and an outer tissue layer, wherein the biocompatible composition comprises one or more of a hydrogel, bio-textile, or polymer (see 804).

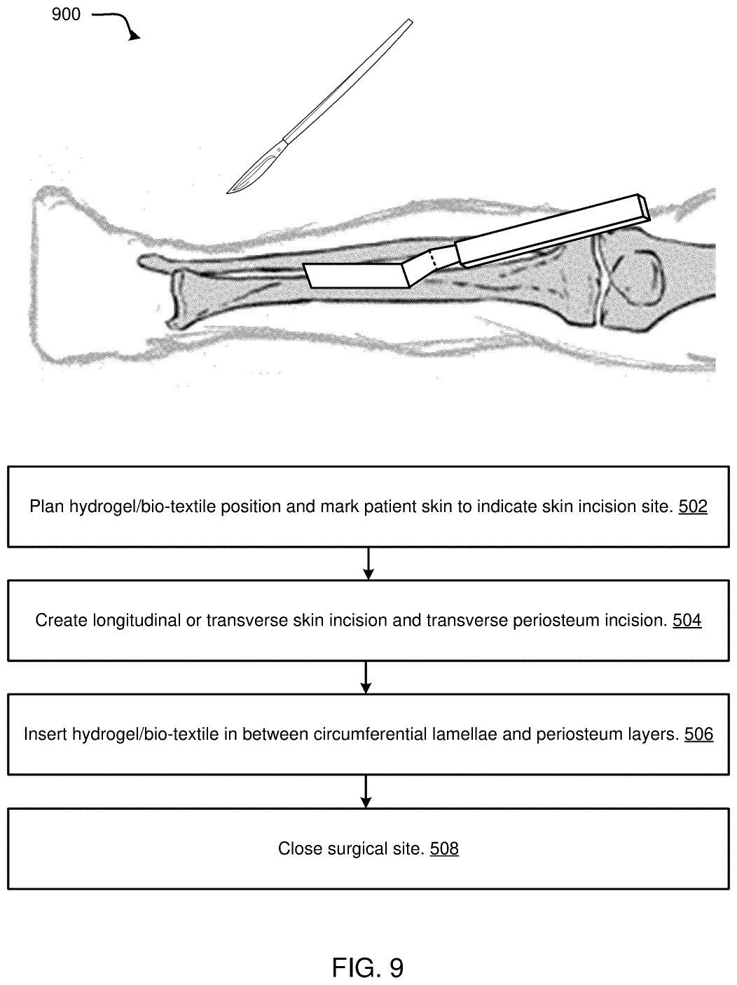

FIG. 9 is a schematic diagram of a method 900 for performing a periosteal distraction surgical procedure with a hydrogel and/or bio-textile. The method 900 includes planning at 902 a hydrogel/bio-textile position and mark the patient skin to indicate the skin incision site. The method 900 includes creating at 904 a longitudinal or transverse skin incision at the surgical site, and further creating a transverse periosteum incision at the surgical site. The method 900 includes inserting at 906 the hydrogel/bio-textile in between the circumferential lamellae and periosteum layers at the surgical site. The method 900 includes closing at 908 the surgical site.

The hydrogels 110 described herein may be optimized for certain vertical displacement, wherein the vertical displacement includes the displacement between an inner tissue layer (see, e.g., the circumferential lamellae 108 or inner soft tissue layer 206, 306) and an outer tissue layer (see, e.g., the periosteum 104, 106 or outer soft tissue layer 204, 304). The vertical displacement of the hydrogel 110 may be from about 0.25 mm to about 1.5 mm per 24-hour period. The vertical displacement of the hydrogel 110 may be from about 0.2 mm to about 2.0 mm per 24-hour period. The vertical displacement of the hydrogel 110 may be from about 0.3 mm to about 1 mm per 24-hour period.

The hydrogels 110 described herein may be provided in various suitable geometry, including a long cylindrical geometry (i.e., wherein a length of the cylinder is longer than a diameter of the cylinder), a short cylindrical geometry (i.e., wherein a diameter of the cylinder is longer than the length of the cylinder), a plate geometry, a polygonal geometry, and so forth. The hydrogels 110 may further be provided in a bead format, wherein a plurality of hydrogel beads is provided within a bag or other vessel.

### EXAMPLES

The following examples pertain to further embodiments.

Table 1, below, illustrates an example formulation of a hydrogel 110 as described herein.

| | Weight/Volume (w/v) |
|---|---|
| | (milligrams of component per 100 |
| Component | milliliters of solution) |
| Sodium alginate | from about 1 w/v to about 5 w/v |
| Calcium chloride | from about 0.1 w/v to about 2 w/v |
| Sodium chloride | from about 8 w/v to about 25 w/v |
| Phosphate buffered | from about 70 w/v to about 120 w/v |
| saline | |

Table 2, below, illustrates an example formulation of a hydrogel 110 as described herein.

| | Weight/Volume (w/v) |
|---|---|
| | (milligrams of component per 100 |
| Component | milliliters of solution) |
| Chitosan | from about 0.5 w/v to about 4 w/v |
| Glycerol phosphate | from about 1 w/v to about 5 w/v |
| Mannitol | from about 5 w/v to about 30 w/v |

Table 3, below, illustrates an example swelling profile for a hydrogel 110 as described herein.

| Day Post Insertion | Optimal Swelling Range |
|---|---|
| 1 | 100-200% initial volume |
| 2 | 150-250% initial volume |
| 3 | 200-300% initial volume |
| 4 | 250-350% initial volume |
| 5 | 300-400% initial volume |
| 6 | 350-450% initial volume |
| 7 | 300-400% initial volume |
| 8-9 | 350-450% initial volume (Maximum) |

Table 4, below, illustrates an example swelling profile for a hydrogel 110 as described herein.

| Day Post Insertion | Optimal Swelling Range |
|---|---|
| 1 | 100-150% initial volume |
| 2 | 150-200% initial volume |
| 3-4 | 200-250% initial volume |
| 5-6 | 250-300% initial volume |
| 7-8 | 300-350% initial volume |
| 9-10 | 350-400% initial volume |
| 11-12 | 400-450% initial volume |
| 13-14 | 450-500% initial volume (Maximum) |

Example 1 is a method. The method includes inserting a biocompatible composition in between an inner tissue layer and an outer tissue layer. The method includes providing an effective duration of time to trigger a tissue generation in response to the biocompatible composition being disposed in between the inner tissue layer and the outer tissue layer.

Example 2 is a method as in Example 1, wherein the biocompatible composition comprises a hydrogel.

Example 3 is a method as in any of Examples 1-2, wherein the biocompatible composition comprises a bio-textile.

Example 4 is a method as in any of Examples 1-3, wherein the biocompatible composition comprises a polymer.

Example 5 is a method as in any of Examples 1-4, wherein one or more of the inner tissue layer or the outer tissue layer comprises a soft tissue.

Example 6 is a method as in any of Examples 1-5, wherein one or more of the inner tissue layer or the outer tissue layer comprises one or more of a blood vessel, a lymph vessel, a muscle, a tendon, a ligament, fat, fibrous tissue, a nerve, cartilage, fascia, or a synovial membrane.

Example 7 is a method as in any of Examples 1-6, wherein the inner tissue layer comprises a circumferential lamellae of a bone.

Example 8 is a method as in any of Examples 1-7, wherein the outer tissue layer comprises periosteum.

Example 9 is a method as in any of Examples 1-8, wherein the biocompatible composition comprises the hydrogel, and wherein the hydrogel expands in between the first tissue layer and the second tissue layer to generate an artificial space that triggers the tissue generation response.

Example 10 is a method as in any of Examples 1-9, further comprising placing a periosteal distraction device, wherein the periosteal distraction device is attached to at least one of the inner tissue layer or the outer tissue layer.

Example 11 is a method as in any of Examples 1-10, wherein the hydrogel comprises at least 10 wt. % interstitial fluid.

Example 12 is a method as in any of Examples 1-11, wherein the hydrogel comprises a porous permeable solid that is water insoluble and comprises a three-dimensional network of polymers.

Example 13 is a method as in any of Examples 1-12, wherein the biocompatible composition comprises a physical hydrogel comprising non-covalent bonds.

Example 14 is a method as in any of Examples 1-13, wherein the biocompatible composition comprises a chemical hydrogel comprising covalent cross-link bonds.

Example 15 is a method as in any of Examples 1-14, wherein the biocompatible composition comprises one or more natural polymers or synthetic polymers.

Example 16 is a method as in any of Examples 1-15, wherein the biocompatible composition comprises one or more of hyaluronic acid, chitosan, heparin, alginate, gelatin, or fibrin.

Example 17 is a method as in any of Examples 1-16, wherein the biocompatible composition comprises one or more of polyvinyl alcohol, polyethylene glycol, sodium polyacrylate, acrylate polymers, or copolymers thereof.

Example 18 is a method as in any of Examples 1-17, wherein the biocompatible composition comprises one or more of a mycelium-based textile, an algae-derived fiber, a cellulose-based textile, or a recombinant protein-based textile.

Example 19 is a method as in any of Examples 1-18, wherein the biocompatible composition comprises one or more of polyvinylidene fluoride, polyethylene, polypropylene, polydimethylsiloxane, parylene, polyamide, polytetrafluoroethylene, polymethyl methacrylate, polyimide, or polyurethane.

Example 20 is a method as in any of Examples 1-19, wherein the method is a procedure for periosteal distraction.

Example 21 is a method as in any of Examples 1-20, wherein the method is a procedure for distraction histogenesis of a soft tissue.

Example 22 is a biocompatible hydrogel for performing a distraction histogenesis surgical procedure on a patient. The biocompatible hydrogel includes a polymer and a crosslinking agent. The biocompatible hydrogel reaches equilibrium with surrounding fluids in the patient from seven days to twenty-two days after implantation in the patient.

Example 23 is a biocompatible hydrogel as in Example 22, wherein the biocompatible hydrogel is implanted underneath a periosteum tissue in the patient to trigger one or more of angiogenesis stimulation, release growth factors to improve blood flow, and accelerate healing of chronic wounds in the patient.

Example 24 is a biocompatible hydrogel as in any of Examples 22-23, wherein the biocompatible hydrogel is at a maximum volume when the biocompatible hydrogel reaches equilibrium; wherein the biocompatible hydrogel comprises an initial volume when the biocompatible hydrogel is implanted in the patient; and wherein the maximum volume is from about three times to about six times larger than the initial volume.

Example 25 is a biocompatible hydrogel as in any of Examples 22-24, wherein the maximum volume is from about four times to about five times larger than the initial volume.

Example 26 is a biocompatible hydrogel as in any of Examples 22-25, wherein the biocompatible hydrogel is resorbed by the patient from about ten days to about thirty days after implantation in the patient.

Example 27 is a biocompatible hydrogel as in any of Examples 22-26, wherein the biocompatible hydrogel is resorbed by the patient from about ten days to about sixteen days after implantation in the patient.

Example 28 is a biocompatible hydrogel as in any of Examples 22-27, wherein the polymer comprises one or more of alginate, chitosan, hyaluronic acid, cellulose, agarose, or pectin.

Example 29 is a biocompatible hydrogel as in any of Examples 22-28, wherein the polymer comprises polyethylene glycol.

Example 30 is a biocompatible hydrogel as in any of Examples 22-29, wherein the polymer comprises one or more of an acrylic polymer or a methacrylic polymer.

Example 31 is a biocompatible hydrogel as in any of Examples 22-30, wherein the polymer comprises a synthetic vinyl polymer.

Example 32 is a biocompatible hydrogel as in any of Examples 22-31, wherein the biocompatible hydrogel is disposed within a syringe for surgical implantation into the patient.

Example 33 is a biocompatible hydrogel as in any of Examples 22-32, wherein the biocompatible hydrogel comprises a cylindrical geometry.

Example 34 is a biocompatible hydrogel as in any of Examples 22-33, wherein the biocompatible hydrogel comprises a cylindrical geometry; and wherein a diameter of the cylindrical geometry is from about three millimeters to about fifteen millimeters when the biocompatible hydrogel is implanted in the patient.

Example 35 is a biocompatible hydrogel as in any of Examples 22-34, wherein the biocompatible hydrogel comprises a cylindrical geometry; and wherein a length of the cylindrical geometry is from about twenty millimeters to about two-hundred millimeters when the biocompatible hydrogel is implanted in the patient.

Example 36 is a biocompatible hydrogel as in any of Examples 22-35, wherein the biocompatible hydrogel is implanted underneath a periosteum tissue in the patient; and wherein the biocompatible hydrogel swells to cause vertical displacement of the periosteum tissue at a rate of about 0.25 millimeters to about 1.5 millimeters per twenty-four period.

Example 37 is a biocompatible hydrogel as in any of Examples 22-36, further comprising a biological growth factor.

Example 38 is a biocompatible hydrogel as in any of Examples 22-37, further comprising a pharmaceutical, wherein the biocompatible hydrogel functions as a modified-release matrix for the pharmaceutical.

Example 39 is a biocompatible hydrogel as in any of Examples 22-38, wherein the biocompatible hydrogel degrades in response to an adjustment in pH in the surrounding fluids in the patient.

Example 40 is a biocompatible hydrogel as in any of Examples 22-39, wherein the biocompatible hydrogel degrades in response to an adjustment in temperature in the surrounding fluids in the patient.

Example 41 is a biocompatible hydrogel as in any of Examples 22-39, wherein the biocompatible hydrogel is implanted into a patient according to any of the method steps of any of Examples 1-21.

The foregoing description has been presented for purposes of illustration. It is not exhaustive and does not limit the invention to the precise forms or embodiments disclosed. Modifications and adaptations will be apparent to those skilled in the art from consideration of the specification and practice of the disclosed embodiments. For example, components described herein may be removed and other components added without departing from the scope or spirit of the embodiments disclosed herein or the appended claims.

Other embodiments will be apparent to those skilled in the art from consideration of the specification and practice of the disclosure disclosed herein. It is intended that the specification and examples be considered as exemplary only, with a true scope and spirit of the invention being indicated by the following claims.

What is claimed is:

1. A biocompatible hydrogel for performing a distraction histogenesis surgical procedure on a patient, the biocompatible hydrogel comprising:
a polymer; and
a crosslinking agent;
wherein the biocompatible hydrogel reaches equilibrium with surrounding fluids in the patient from seven days to twenty-two days after implantation in the patient.

2. The biocompatible hydrogel of claim 1, wherein the biocompatible hydrogel is implanted underneath a periosteum tissue in the patient to trigger one or more of angiogenesis stimulation, release growth factors to improve blood flow, and accelerate healing of chronic wounds in the patient.

3. The biocompatible hydrogel of claim 1, wherein the biocompatible hydrogel is at a maximum volume when the biocompatible hydrogel reaches equilibrium;
wherein the biocompatible hydrogel comprises an initial volume when the biocompatible hydrogel is implanted in the patient; and
wherein the maximum volume is from about three times to about six times larger than the initial volume.

4. The biocompatible hydrogel of claim 3, wherein the maximum volume is from about four times to about five times larger than the initial volume.

5. The biocompatible hydrogel of claim 1, wherein the biocompatible hydrogel is resorbed by the patient from about ten days to about thirty days after implantation in the patient.

6. The biocompatible hydrogel of claim 1, wherein the biocompatible hydrogel is resorbed by the patient from about ten days to about sixteen days after implantation in the patient.

7. The biocompatible hydrogel of claim 1, wherein the polymer comprises one or more of alginate, chitosan, hyaluronic acid, cellulose, agarose, or pectin.

8. The biocompatible hydrogel of claim 1, wherein the polymer comprises polyethylene glycol.

9. The biocompatible hydrogel of claim 1, wherein the polymer comprises one or more of an acrylic polymer or a methacrylic polymer.

10. The biocompatible hydrogel of claim 1, wherein the polymer comprises a synthetic vinyl polymer.

11. The biocompatible hydrogel of claim 1, wherein the biocompatible hydrogel is disposed within a syringe for surgical implantation into the patient.

12. The biocompatible hydrogel of claim 1, wherein the biocompatible hydrogel comprises a cylindrical geometry.

13. The biocompatible hydrogel of claim 1, wherein the biocompatible hydrogel comprises a cylindrical geometry; and
wherein a diameter of the cylindrical geometry is from about three millimeters to about fifteen millimeters when the biocompatible hydrogel is implanted in the patient.

14. The biocompatible hydrogel of claim 1, wherein the biocompatible hydrogel comprises a cylindrical geometry; and
wherein a length of the cylindrical geometry is from about twenty millimeters to about two-hundred millimeters when the biocompatible hydrogel is implanted in the patient.

15. The biocompatible hydrogel of claim 1, wherein the biocompatible hydrogel is implanted underneath a periosteum tissue in the patient; and
wherein the biocompatible hydrogel swells to cause vertical displacement of the periosteum tissue at a rate of about 0.25 millimeters to about 1.5 millimeters per twenty-four period.

16. The biocompatible hydrogel of claim 1, further comprising a biological growth factor.

17. The biocompatible hydrogel of claim 1, further comprising a pharmaceutical, wherein the biocompatible hydrogel functions as a modified-release matrix for the pharmaceutical.

18. The biocompatible hydrogel of claim 1, wherein the biocompatible hydrogel degrades in response to an adjustment in pH in the surrounding fluids in the patient.

19. The biocompatible hydrogel of claim 1, wherein the biocompatible hydrogel degrades in response to an adjustment in temperature in the surrounding fluids in the patient.

20. A method comprising:
implanting a biocompatible hydrogel in a patient to perform a distraction histogenesis surgical procedure on the patient, wherein the biocompatible hydrogel comprises:
a polymer; and
a crosslinking agent;
wherein the biocompatible hydrogel reaches equilibrium with surrounding fluids in the patient from seven days to twenty-two days after implantation in the patient.
